(19) United States
(12) Patent Application Publication
(10) Pub. No.: US 2005/0283045 A1
(43) Pub. Date: Dec. 22, 2005

(54) Capsule medical instrument with oxygen generator

(75) Inventors: Akira Yamamoto, Tokyo (JP); Masafumi Kanazawa, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/143,604

(22) Filed: Jun. 3, 2005

(30) Foreign Application Priority Data
Jun. 16, 2004 (JP) 2004-178728

(51) Int. Cl. A61K 38/43; A61K 9/22; A61K 33/40

(52) U.S. Cl. 600/102; 604/890.1; 424/94.1; 424/616

(57) ABSTRACT

A capsule medical instrument having a capsule which can be swallowed into a body cavity and an oxygen generator provided within the capsule, the oxygen generator including a reservoir for hydrogen peroxide solution, a catalyst which accelerates a rate of decomposition reaction of the hydrogen peroxide solution into oxygen and water, a control device for controlling the rate of decomposition reaction of the hydrogen peroxide with the catalyst, and a discharge system which discharges the oxygen and the water produced by the decomposition reaction.

### BACKGROUND OF THE INVENTION

1. Field of the Invention

The present invention relates to a capsule medical instrument and, particularly, it relates to a capsule medical instrument having an oxygen generator.

2. Description of the Related Art

A capsule endoscope has been proposed and used as a capsule medical instrument which can be swallowed into a body cavity. Since the capsule endoscope constitutes an independent system, one problem that needs to be solved is providing an air supply method. In an endoscope, air (harmless gas) must be discharged and fed to expand a predetermined portion of the body cavity. If an air cylinder (pressurized gas cylinder) is provided, the endoscope must be pressure resistant, thus resulting in an increase in size. In a liquid medicament delivery capsule which is adapted to supply liquid medicament to a target portion of the body cavity, a power source must be provided in order to feed the liquid medicament.

### SUMMARY OF THE INVENTION

The present invention provides a capsule medical instrument having an oxygen generator, in which air can be easily fed without providing an air cylinder.

The present invention has been devised based on the conception that if hydrogen peroxide solution is provided, which can be decomposed into oxygen and water with the help of a catalyst which accelerates the decomposition reaction, the necessary amount of oxygen and the necessary amount of water can be obtained at a desired time by controlling the rate of decomposition reaction of the hydrogen peroxide solution with the catalyst.

According to an aspect of the present invention, a capsule medical instrument is provided, having a capsule which can be swallowed into a body cavity and an oxygen generator provided within the capsule, the oxygen generator including a reservoir for hydrogen peroxide solution, a catalyst which accelerates a rate of decomposition reaction of the hydrogen peroxide solution into oxygen and water, a control device for controlling the rate of decomposition reaction of the hydrogen peroxide with the catalyst, and a discharge system which discharges the oxygen and the water produced by the decomposition reaction.

It is desirable for the control device to control an amount of exposure of a contact surface area of the catalyst with respect to the hydrogen peroxide solution.

It is desirable for the control device to control the amount of hydrogen peroxide solution to be fed to the catalyst.

It is desirable for the medical instrument to include a capsule endoscope; and wherein the oxygen and the water discharged through the discharged system are discharged to the body cavity of a patient's body.

It is desirable for the medical instrument to include a liquid medicament delivery capsule which delivers a liquid medicament into a patient's body. The oxygen and the water discharged through the discharge system are used to discharge the liquid medicament from a liquid medicament reservoir provided in the liquid medicament delivery capsule.

It is desirable for the catalyst to be made of manganese dioxide or a catalase.

The present disclosure relates to subject matter contained in Japanese Patent Application No. 2004-178728 (filed on Jun. 16, 2004) which is expressly incorporated herein in its entirety.

### BRIEF DESCRIPTION OF THE DRAWINGS

The present invention will be described below in detail with reference to the accompanying drawings in which:

FIG. 1 is a conceptual sectional view of an embodiment of a capsule endoscope according to the present invention;

FIG. 2 is a conceptual view of a first embodiment of an oxygen generator provided in the capsule endoscope shown in FIG. 1;

FIG. 3 is a view viewed from the direction of the arrows II in FIG. 2;

FIG. 4 is a conceptual view of a second embodiment of an oxygen generator provided in the capsule endoscope shown in FIG. 1;

FIG. 5 is a conceptual view of a third embodiment of an oxygen generator provided in the capsule endoscope shown in FIG. 1;

FIG. 6 is a conceptual sectional view of a first embodiment of a liquid medicament delivery capsule according to the present invention; and

FIG. 7 is a conceptual sectional view of a second embodiment of a liquid medicament delivery capsule according to the present invention.

### DESCRIPTION OF THE PREFERRED EMBODIMENT

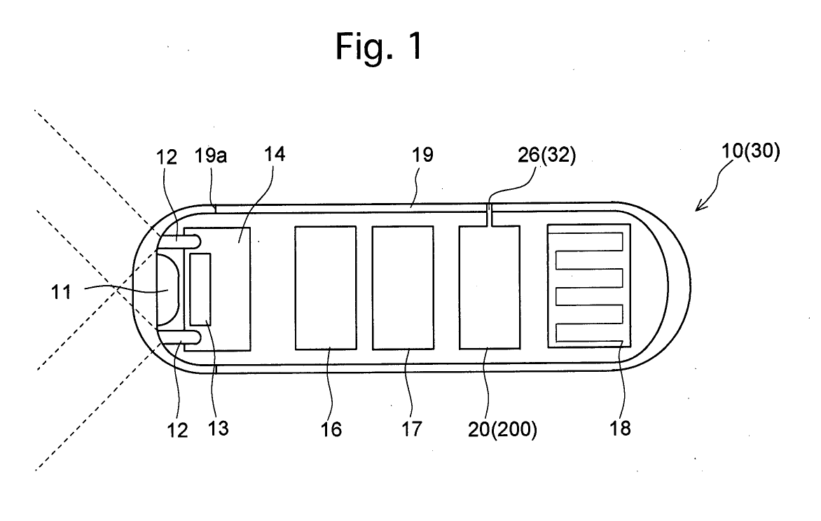

A capsule endoscope 10 according to the present invention includes an objective optical system 11, a signal processor/light emitter 14 having a light emitting diode (LED) 12 which emits light to illuminate a patient's body cavity and having a solid-state image pickup device 13, a signal transmitter/receiver circuit 16, a battery 17 constituting a driving power source, a signal transmitter/receiver antenna 18, and an oxygen generator 20, in that order from the front of the capsule endoscope 10 (left side in FIG. 1), and which are accommodated in a sealed capsule 19, sealed in a water-tight fashion.

In the capsule endoscope 10, an image of an examined portion which is illuminated with light emitted from the LED 12 and which is viewed through a transparent cover 19a is formed through the objective optical system 11 and picked up by the solid-state image pickup device 13. The image signal output from the solid state image pickup device 13 is modulated and amplified by the signal transmitter/receiver circuit 16 and is transmitted to the outside of the patient's body through the signal transmitter/receiver antenna 18. The sealed capsule 19 has a hemispherical shape at the front and rear ends thereof, so that the sealed capsule is generally in the form of a smoothly rounded cylinder. The transparent hemispherical cover 19a is provided at the front end of the sealed capsule 19. The sealed capsule 19 is provided with a filter (not shown) which permits air to pass therethrough but does not permit water to pass, so that there is substantially no difference in air pressure between the inside and outside of the sealed capsule 19.

This air-permeable/water non-permeable filter can be made of, for example, a hydrophobic sheet with sub-micron holes. Since liquid particles are approximately 100 μm, the sub-micron holes of the hydrophobic sheet do not permit liquid to pass therethrough due to the diameter of the sub-micron holes being smaller than the diameter of the liquid particles. However, gas particles are sub nanometers in size, and accordingly, can pass through the sub-micron holes of the hydrophobic sheet. Hence, substantially no pressure difference occurs between the outside and inside of the sealed capsule 19, while reserving the sealing efficiency of the sealed capsule 19.

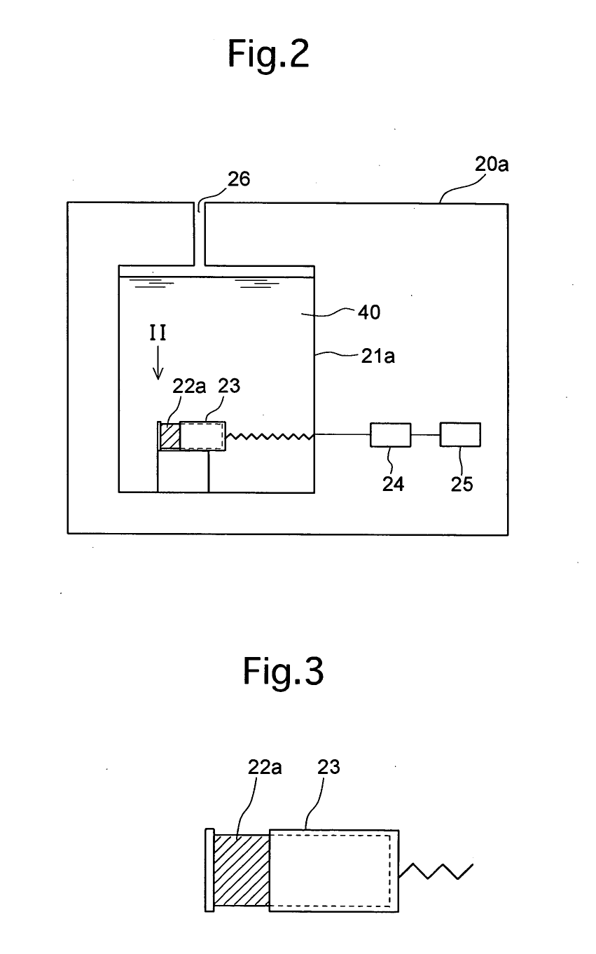

FIGS. 2, 4 and 5 show first through third embodiments of the oxygen generator 20(20a, 20b and 20c) and are be discussed below.

### Embodiment 1 of the Oxygen Generator

The oxygen generator 20a is provided with a hydrogen peroxide solution reservoir 21a for hydrogen peroxide solution, as shown in FIG. 2. The hydrogen peroxide solution reservoir 21a is provided with a catalyst 22a. The hydrogen peroxide solution reservoir 21a is filled with hydrogen peroxide solution 40 and is provided with a discharge pipe 26 connected to the reservoir (FIG. 1). The catalyst 22a is in the form of a plate of manganese dioxide or catalase. The exposed surface area of the catalyst can be varied by a cover member 23 which is moved by an actuator 24. The actuator 24 can be, for example, an electromagnetic actuator.

When the signal transmitter/receiver antenna 18 in the capsule endoscope 10 receives a radio drive signal, the cover member 23 is moved by the actuator 24 via the signal transmitter/receiver circuit 16 and a driver (control device) 25 to vary the exposed surface area of the catalyst 22a. Namely, the oxygen generator 20a in this embodiment is of the type in which the rate of decomposition reaction is controlled by controlling the contact surface area (exposed surface area) of the catalyst 22a.

The capsule endoscope 10 constructed as above is swallowed in a patient's body with the hydrogen peroxide solution reservoir 21a being filled with hydrogen peroxide solution and with the catalyst 22a being covered by the cover member 23. In this state, sufficient oxygen is not generated. If it is necessary to expand the patient's body cavity during observing the examined portion via the image formed through the objective optical system 11, picked up by the solid state image pickup device 13, and transmitted via the signal transmitter/receiver circuit 16 and the signal transmitter/receiver antenna 18, of the capsule endoscope 10, the operator (user) sends a radio drive signal. When the radio drive signal is received by the signal transmitter/receiver antenna 18, the cover member 23 is moved via the signal transmitter/receiver circuit 16, the driver 25, and the actuator 24, to thereby expose the catalyst 22a. When the catalyst 22a is exposed, the hydrogen peroxide solution 40 in the hydrogen peroxide solution reservoir 21 is in contact with the catalyst 22a, so that the rate of decomposition reaction of the hydrogen peroxide solution into water and oxygen is accelerated. The oxygen thus produced is discharged through the discharge pipe 26 to the outside of the capsule endoscope 10 to expand the patient's body cavity. In addition to oxygen, water or hydrogen peroxide solution (liquid) may be discharged from the discharge pipe 26. The hydrogen peroxide solution is a medicament used as a disinfectant and, accordingly, even if a small amount of hydrogen peroxide solution is discharged into the body cavity, there is no harmful effect. In the portion in which bleeding occurs, the hydrogen peroxide solution discharged into the body cavity reacts on the catalase contained in the blood to generate oxygen and, thus, the discharged hydrogen peroxide is useful to identify the bleeding portion and contributes to disinfection.

### Embodiment 2 of the Oxygen Generator

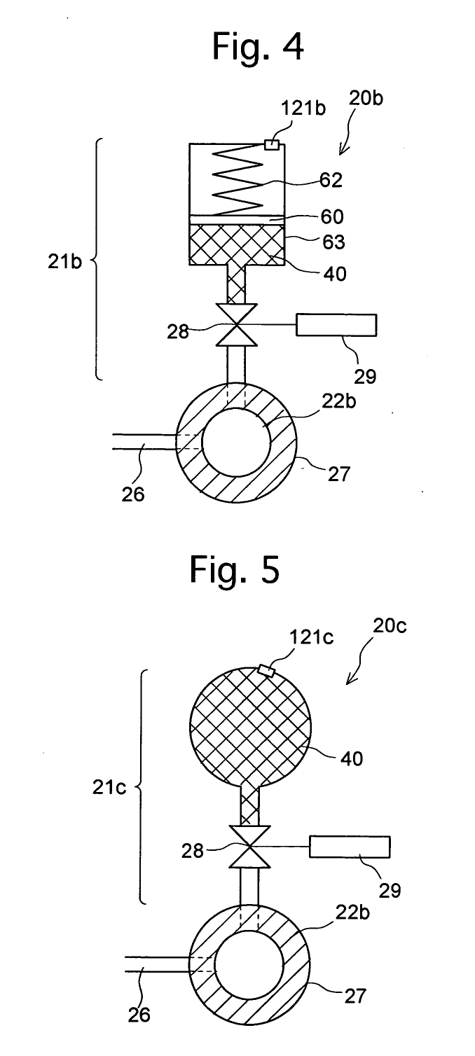

FIG. 4 shows a second embodiment of an oxygen generator 20b in which the amount of hydrogen peroxide solution to be supplied to a catalyst 22b is controlled.

In the oxygen sensor 20b, a control circuit (control device) 29 opens an electromagnetic valve 28 in accordance with instructions of the operator (user), so that the hydrogen peroxide solution 40 reserved in a hydrogen peroxide solution reservoir 21b is supplied to a catalyst 27 having the form of a hollow sphere. Consequently, the decomposition reaction of the hydrogen peroxide solution 40 with the catalyst in the catalyzing ball 27 occurs to produce water and oxygen. The water and oxygen thus produced are discharged from the discharge pipe 26 into the capsule endoscope 10.

The hydrogen peroxide solution reservoir 21b has a movable separation wall (piston) 60 which divides the internal space of the cylinder 63 into two compartments, one of which is provided therein with a spring 62. The other compartment is filled with the hydrogen peroxide solution 40. The cylinder 63 is provided, on the side thereof that is not in contact with the hydrogen peroxide solution, with an air-permeable/water non-permeable filter 121b. The spring 62 continuously biases the piston in a direction to discharge the hydrogen peroxide solution, so that when the electromagnetic valve 28 is open, water or oxygen cannot be returned to the hydrogen peroxide solution reservoir 21b. The separation wall 60 is freely movable and is continuously biased by the spring 62, so that when the electromagnetic valve 28 is opened, the separation wall 60 is moved to discharge the hydrogen peroxide solution 40 from the hydrogen peroxide solution reservoir 21b. The air-permeable/water non-permeable filter 121b is made of the same material as that provided in the sealed capsule 19 so that the internal pressure and the external pressure of the hydrogen peroxide solution reservoir 21b can be maintained substantially the same as each other.

The catalyzing ball 27 is provided on its entire inner surface with a catalyst (manganese dioxide or catalase) and is provided with a discharge pipe 26 connected thereto, to which the electromagnetic valve 28 is also connected. The discharge pipe 26 is connected to the sealed capsule 19.

The electromagnetic valve 28 selectively connects the hydrogen peroxide solution reservoir 21b and the catalyzing ball 27 and is opened or closed in accordance with the operator's instructions via the control circuit 29. The control circuit 29 operates through the signal transmitter/receiver circuit 16 in accordance with the radio drive signal sent from the operator and received by the signal transmitter/receiver antenna 18 to control the opening and closing operations of the electromagnetic valve 28.

If it is necessary to expand the patient's body cavity during observation of the examined portion by the image formed through the objective optical system 11, picked up by the solid state image pickup device 13, and transmitted via the signal transmitter/receiver circuit 16 and the signal transmitter/receiver antenna 18, of the capsule endoscope 10, the operator (user) sends a radio drive signal accordingly. When the radio drive signal is received by the signal transmitter/receiver antenna 18, the electromagnetic valve 28 is opened via the signal transmitter/receiver circuit 16 and the control circuit 29 to thereby supply the hydrogen peroxide solution 40 to the catalyzing ball 27. When the hydrogen peroxide solution 40 is supplied to the catalyzing ball 27, the decomposition reaction of the hydrogen peroxide solution into water and oxygen takes place due to the catalyst 22b spreading over the entire inner surface of the catalyzing ball 27. The oxygen thus produced is discharged through the discharge pipe 26 to the outside of the capsule endoscope 10 to expand the patient's body cavity. The amount of the hydrogen peroxide solution to be supplied to the catalyzing ball 27 can be controlled by controlling the opening or closing time of the electromagnetic valve 28 in order to obtain a desired amount of oxygen.

### Embodiment 3 of the Oxygen Generator

FIG. 5 shows a third embodiment of the oxygen generator in which the hydrogen peroxide solution reservoir 21b in the oxygen generator 20b is replaced with a balloon reservoir 21c. In this embodiment, the reacting amount of the hydrogen peroxide solution is controlled by controlling the amount of hydrogen peroxide solution to be supplied to the catalyst.

The balloon reservoir 21c is made of a material which is resistant to the hydrogen peroxide solution 40 and which is expandable and retractable. The hydrogen peroxide solution 40 is supplied due to the retraction of the balloon reservoir 21c. The balloon reservoir 21c is provided with an air-permeable/water non-permeable filter 121c. The air-permeable/water non-permeable filter 121c is made of the same material as that of the air-permeable/water non-permeable filter provided in the sealed capsule 19 so as to prevent a pressure difference occurring between the inside and outside of the balloon reservoir 21c. The mechanism to discharge oxygen and water obtained from the hydrogen peroxide solution to the outside of the capsule endoscope 10 in order for an operator to expand the patient's body cavity is the same as that in the oxygen generator 20b.

### Embodiment 1 of the Liquid Medicament Delivery Capsule

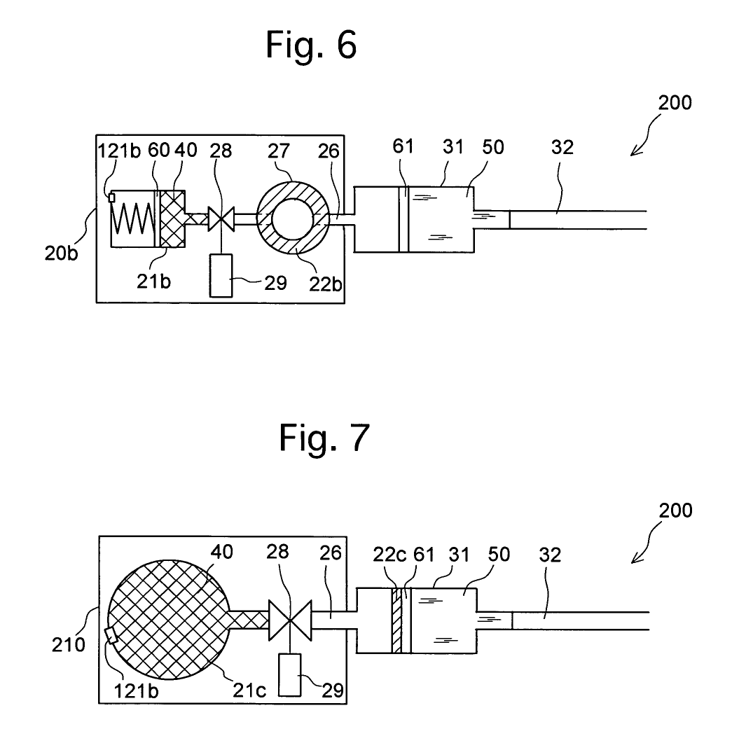

FIG. 6 shows a first embodiment of a liquid medicament delivery capsule 30 according to the present invention. A liquid medicament supply device 200, in place of the oxygen generator 20 shown in FIG. 1, is arranged in the liquid medicament delivery capsule 30. The liquid medicament supply device 200 is provided therein with the oxygen generator 20b, the discharge pipe 26, and a liquid medicament reservoir 31 in which the necessary liquid medicament 50 is enclosed. The discharge pipe 32 connected to the liquid medicament reservoir 31 is connected to and opens out to the liquid medicament delivery capsule 30 (FIG. 1). The oxygen generator 20b supplies the hydrogen peroxide solution 40 to the catalyzing ball 27 in accordance with the operator's instructions. The inside of the liquid medicament reservoir 31 is divided into two compartments by a movable separation wall 60. One of the compartments having the discharge pipe 32 connected thereto is filled with the liquid medicament 50 and the other compartment is connected to the catalyzing ball 27 (FIG. 4) through the discharge pipe 26, through which oxygen and water generated in the catalyzing ball 27 are supplied to the other compartment.

The liquid medicament delivery capsule 30 constructed as above is swallowed in a patient's body, for example, during observation using a contrast medium. When the liquid medicament delivery capsule 30 reaches a predetermined portion of the patient's body cavity, if the operator (user) sends a radio drive signal to discharge the liquid medicament 50, the electromagnetic valve 28 is opened via the signal transmitter/receiver antenna 18, the signal transmitter/receiver circuit 16 and the control circuit 29, so that the hydrogen peroxide solution 40 is fed to the catalyzing ball 27. When the hydrogen peroxide solution 40 is introduced into the catalyzing ball 27, the decomposition reaction of the hydrogen peroxide solution into water and oxygen takes place. The oxygen and water thus produced are discharged through the discharge pipe 26 into the liquid medicament reservoir 31 and are used to discharge the liquid medicament 50 from the liquid medicament reservoir 31. An increase in the amount of oxygen and water causes an increase in the internal pressure of the liquid medicament reservoir 31. Consequently, the pressure is applied to the movable separation wall 61 to thereby feed the liquid medicament 50, the amount of which corresponds to the increased internal pressure from the liquid medicament reservoir 31 into the body cavity through the discharge pipe 32. Since the pressure applied to the liquid medicament reservoir 31 can be controlled in accordance with the time in which the electromagnetic valve 28 is open (the amount of oxygen to be produced), the amount of the liquid medicament 50 to be supplied can be controlled in accordance with the opening time of the electromagnetic valve 28.

The oxygen generator 20a (FIG. 3) or the oxygen generator 20c (FIG. 5) can be used instead of the oxygen generator 20b.

### Embodiment 2 of the Liquid Medicament Delivery Capsule

FIG. 7 shows a second embodiment of the liquid medicament delivery capsule 30 according to the present invention. A liquid medicament supply device 200, instead of the oxygen generator 20 shown in FIG. 1, is arranged in the liquid medicament delivery capsule 30. The liquid medicament supply device 200 is provided therein with a liquid medicament reservoir 31 in which the necessary liquid medicament 50 is enclosed, a hydrogen peroxide solution supply device 210 and a discharge pipe 26. In the liquid medicament reservoir 31, the movable separation wall 61 is provided, on the surface thereof adjacent to the balloon reservoir 21c, with a catalyst 22c. The hydrogen peroxide solution supply device 210 is provided with the balloon reservoir 21c, the electromagnetic valve 28, and the control circuit 29, and supplies the hydrogen peroxide solution 40 to the liquid medicament reservoir 31 through the discharge pipe 26, in accordance with the operator's instructions.

If the operator (user) sends a radio drive signal to discharge the liquid medicament 50, the electromagnetic valve 28 is opened via the signal transmitter/receiver antenna 18, the signal transmitter/receiver circuit 16 and the control circuit 29 provided in the hydrogen peroxide solution supply device 210, so that the hydrogen peroxide solution 40 is fed to the liquid medicament reservoir 31 through the discharge pipe 26. When the hydrogen peroxide solution 40 is introduced into the liquid medicament reservoir 31, decomposition reaction of the hydrogen peroxide solution into water and oxygen occurs due to the catalyst 22c provided on the surface of the movable separation wall 61 located adjacent to the balloon reservoir 21c. Consequently, oxygen and water are produced. An increase in the amount of oxygen and water causes an increase in the internal pressure of the liquid medicament reservoir 31. As a result, the pressure is applied to the movable separation wall 61 to thereby feed the liquid medicament 50 (the amount of which corresponds to the increased internal pressure) from the liquid medicament reservoir 31 into the body cavity through the discharge pipe 32.

In the hydrogen peroxide solution supply device 210, the balloon reservoir 21c can be replaced with the hydrogen peroxide solution reservoir 21b. The oxygen generator 20a can be used instead of the hydrogen peroxide solution supply device 210. In the case where the oxygen generator 20a is used, no catalyst 22c is necessary.

Although the present invention is applied to a capsule endoscope or a liquid medicament delivery capsule in the embodiments mentioned above, the present invention can be applied to a capsule medical instrument in general in which it is necessary to generate oxygen in a patient's body.

Obvious changes may be made in the specific embodiments of the present invention described herein, such modifications being within the spirit and scope of the invention claimed. It is indicated that all matter contained herein is illustrative and does not limit the scope of the present invention.

1. A capsule medical instrument having a capsule which can be swallowed into a body cavity and an oxygen generator provided within said capsule, said oxygen generator comprising:
a reservoir for hydrogen peroxide solution;
a catalyst which accelerates a rate of decomposition reaction of the hydrogen peroxide solution into oxygen and water;
a control device for controlling the rate of decomposition reaction of the hydrogen peroxide with the catalyst; and
a discharge system which discharges the oxygen and the water produced by said decomposition reaction.

2. The capsule medical instrument according to claim 1, wherein said control device controls an amount of exposure of a contact surface area of the catalyst with respect to the hydrogen peroxide solution.

3. The capsule medical instrument according to claim 1, wherein said control device controls the amount of hydrogen peroxide solution to be fed to the catalyst.

4. The capsule medical instrument according to claim 1, wherein said medical instrument comprises a capsule endoscope; and wherein the oxygen and the water discharged through the discharged system are discharged to the body cavity of a patient's body.

5. The capsule medical instrument according to claim 1, wherein the medical instrument comprises a liquid medicament delivery capsule which delivers a liquid medicament into a patient's body; and
wherein the oxygen and the water discharged through the discharge system are used to discharge the liquid medicament from a liquid medicament reservoir provided in the liquid medicament delivery capsule.

6. The capsule medical instrument, according to claim 1, wherein said catalyst is made of one of manganese dioxide and a catalase.
